(19) United States
(12) Patent Application Publication
(10) Pub. No.: US 2005/0186171 A1
(43) Pub. Date: Aug. 25, 2005

(54) Skin cream

(76) Inventor: Bernyce Alpert Winick, Woodmere, NY (US)

Correspondence Address: Philip M. Weiss;Weiss & Weiss, Suite 201, 310 Old Country Road, Garden City, NY, 11530, US

(21) Appl. No.: 10/945,343

(22) Filed: Sep. 20, 2004

Related U.S. Application Data
(60) Provisional application No. 60/546,797, filed on Feb. 23, 2004.

(51) Int. Cl. A61K 7/06; A61K 35/78

(52) U.S. Cl. 424/74; 424/757; 424/744

(57) ABSTRACT

A skin cream which relates to a combination of natural oils and healing ointments that feed, heal and nourish dry mature skin as they lubricate and rejuvenate, reducing the formation of wrinkles.

### RELATED APPLICATIONS

The present application claims priority to provisional patent application Ser. No. 60/546,797, filed Feb. 23, 2004.

### FIELD OF THE INVENTION

The present invention relates to a combination of natural oils and healing ointments that feed, heal, and nourish dry mature skin as they lubricate and rejuvenate, reducing the formation of wrinkles.

### BACKGROUND OF THE INVENTION

U.S. Pat. Nos. 6,551,607 and 6,521,241 relate to a method of sequestering skin irritants with a skin irritant sequestering composition comprising a substrate, a hydrophilic skin irritant sequestering agent and a hydrophobic skin irritant sequestering agent.

U.S. Pat. No. 6,517,848 relates to a method for sequestration of skin irritants with absorbent article compositions. The composition comprises an absorbent article having disposed thereon a skin irritant sequestering effective amount of an unmodified particulate skin irritant sequestering agent and a lipophilic skin health benefit agent. A lipophilic skin health benefit agent can be stearic acid, isoparrafin, petrolatum, and a combination thereof.

U.S. Pat. No. 6,534,074 relates to a superior skin barrier enhancing body facing material, such as a body sideliner on an absorbent article, can be made by applying, on the outer surface of the body facing material, a lipid-enriched hydrophobic composition comprising a natural fat or oil, a sterol, or sterol derivative, an emulsifying surfactant, a humectant, an emollient, a wax, and a viscosity enhancer, and thereafter resolidifying the composition to form a distribution of solid composition on the outer surface of the body facing material.

U.S. Pat. No. 6,485,733 relates to an absorbent article composition for sequestering skin irritants. The article has disposed thereon a skin irritant sequestering effective amount of an unmodified particulate skin irritant sequestering agent and a lipophilic skin health benefit agent. An unmodified particulate skin irritant sequestering agent can be a clay, such as bentonite or laponite unmodified by organic amphiphilic compounds. A lipophilic skin health benefit agent can be stearic acid, isoparrafin, petrolatum, and a combination thereof.

U.S. Pat. No. 6,342,208 relates to an oil-water emulsion for application on a skin surface. The emulsion comprises an oily phase and an aqueous phase. The oily phase comprises a first lipid of vegetable or animal origin. The emulsion is stabilized by containing at least one surfactant/emulsifier.

U.S. Pat. No. 6,312,714 relates to a cosmetic composition for rejuvenating the appearance of skin with reduced or minimal potential for skin irritation, in the form of a lotion, crème, solution or gel, or applied onto a carrier, includes about 0.5 to 50 wt. % in an aqueous, alcoholic or aqueous-alcoholic solution of a solvent-free, high molecular weight terpolymer of methyl vinyl ethermaleic anhydride-isobutylene, said composition having a pH of 1.5 to 5.

U.S. Pat. No. 6,287,581 relates to a superior skin barrier enhancing body facing material, such as a body side liner on an absorbent article, can be made by applying, on the outer surface of the body facing material, a lipid-enriched hydrophobic composition comprising a natural fat or oil, a sterol or sterol derivative, an emulsifying surfactant, a humectant, an emollient, a wax, and a viscosity enhancer, and thereafter resolidifying the composition to form a distribution of solid composition on the outer surface of the body facing material.

U.S. Pat. No. 5,985,809 relates to a personal cleansing composition comprising: (a) from about 1% to about 30% by weight of a dispersed oil phase comprising at least one nonocculsive liquid polyol fatty acid polyester containing at least four fatty acid ester groups wherein the polyol moiety is selected from sugars and sugar alcohols containing from about 4 to about 8 hydroxyl groups, and wherein each carboxylic acid moiety has from about 8 to about 22 carbon atoms and wherein the liquid polyol fatty acid polyester has a complete melting point of less than about 30° C.; and (b) from about 5% to about 30% by weight of water-soluble surfactant selected from anionic, nonionic, zwitterionic and amphoteric surfactants and mixtures thereof.

U.S. Pat. No. 5,942,479 relates to a personal cleansing composition comprising: (a) from about 1% to about 30% by weight of a dispersed oil phase comprising a first oil component having an oil/surfactant solution interfacial tension (IFT) of greater than about 1.0 dynes/cm and a second oil component having an oil/surfactant solution interfacial tension (IFT) in the range of from about 0.1 to about 1.0 dynes/cm; and (b) from about 5% to about 30% by weight of water-soluble surfactant selected from anionic, nonionic, zwitterionic and amphoteric surfactants and mixtures thereof; and (c) water; and wherein the ratio of first to second dispersed oil component is in the range from about 20:1 to about 1:20.

U.S. Pat. No. 5,910,472 relates to a liquid personal cleansing composition comprising: (a) from about 5% to about 60% by weight of water-soluble surfactant selected from anionic, nonionic and amphoteric surfactants and mixtures thereof; and (b) from about 0.01% to about 10% by weight of a hydrophobically modified nonionic cellulose ether selected from C₁₄-C₁₈ alkyl and alkenyl modified, hydroethyl cellulose ethers having a degree of nonionic substitution in the range of from about 2.2 to about 2.8 and a degree of hydrophobic substitution in the range of from about 0.4% to about 0.6% by weight; and (c) water, and wherein the composition displays a shear stress of about 150 Pa at a shear rate in the range from about 400 s⁻¹ to about 600 s⁻¹ at 25° C.

U.S. Pat. No. 5,736,128 relates to a cosmetic composition for rejuvenating the appearance of skin with reduced or minimal potential for skin irritation, in the form of a lotion, crème, solution or gel, includes an aqueous, alcoholic or aqueous-alcoholic solution of a polymer having a carboxylic acid functionality, the solution having a pH of 1.5 to 5.

### SUMMARY OF THE INVENTION

The present invention relates to a combination of natural oils and healing ointments that feed, heal and nourish dry mature skin as they lubricate and rejuvenate, reducing the formation of wrinkles.

The present invention relates to a Day Crème composition comprising:
AUBREY ULTRA
A AND D OINTMENT
SHEA BUTTER
MIXED OILS including AVOCADO OIL, GRAPE SEED OIL, SWEET ALMOND OIL, ALOE VERA EXTRACT IN SOYBEAN OIL and JOJOBA OIL
GLYCERIN
EFA CAPSULE (ESSENTIAL FATTY ACIDS) including BORAGE OIL, FISH OIL, GLYCERIN and MIXED OILS which comprise AVOCADO, GRAPESEED, SWEET ALMOND, ALOE VERA, JOJOBA; and
ALOE VERA

The present invention relates to a Night Crème composition comprising:
LANOLIN TUBE EMOLLIENT—WHITE
LANOLIN ANHYDROUS—YELLOW PURE
ALOE VERA
JOJOBA OIL
GLYCERIN
ALMOND OIL
VITAMIN A AND D
VASELINE
A AND D OINTMENT
DESITIN
AUBREY ULTRA
AQUAFOR
SHEA BUTTER
AVOCADO OIL
E GEMS LIQUID
EFA (ESSENTIAL FATTY ACIDS) capsules including FLAX SEED OIL, BORAGE SEED OIL, FISH OIL, GLYCERIN and MIXED OILS COMPRISING AVOCADO, GRAPE SEED, SWEET ALMOND, ALOE VERA, JOJOBA;
PRIMROSE OIL AND
VITAMIN E

It is an object of the present invention for the Night Crème to comprise, cod liver oil and zinc oxide.

### DETAILED DESCRIPTION OF THE INVENTION

Day Creme

In an embodiment the day creme comprises:
approximately 6 oz. of AUBREY ULTRA;
approximately 0.75 oz. of A and D OINTMENT;
approximately 3 full tablespoons SHEA BUTTER;
approximately 4-8 drops of MIXED OILS including AVOCADO OIL, GRAPESEED OIL, SWEET ALMOND OIL, ALOE VERA EXTRACT IN SOYBEAN OIL and JOJOBA OIL;
approximately 1 tablespoon of GLYCERIN;
approximately one EFA capsule (ESSENTIAL FATTY ACIDS) including BORAGE OIL, FISH OIL, GLYCERIN and MIXED OILS which comprise AVOCADO, GRAPESEED, SWEET ALMOND, ALOE VERA, JOJOBA; and
approximately 1½ tablespoons ALOE VERA.

In a preferred embodiment the Aubrey Ultra is Aubrey Ultra 15.

Night Creme

In an embodiment the night creme comprises:
approximately 1 oz. of LANOLIN EMOLLIENT—WHITE;
approximately 3 teaspoons of LANOLIN ANHYDROUS—YELLOW PURE;
approximately 2 oz. ALOE VERA;
approximately one tablespoon JOJOBA OIL;
approximately one tablespoon GLYCERIN;
approximately one tablespoon ALMOND OIL;
approximately six capsules VITAMIN A and D;
approximately 2.5 oz. VASELINE;
approximately 3.75 oz. A and D OINTMENT;
approximately 1.5 oz. DESITIN;
approximately 10 oz. AUBREY ULTRA 15;
approximately 3 tablespoons SHEA BUTTER;
approximately one tablespoon AVOCADO OIL;
approximately one tablespoon E GEMS LIQUID;
approximately two capsules EFA (ESSENTIAL FATTY ACIDS) capsules including FLAX SEED OIL, BORAGE SEED OIL, FISH OIL, GLYCERIN and MIXED OILS COMPRISING AVOCADO, GRAPE SEED, SWEET ALMOND, ALOE VERA, JOJOBA;
approximately four capsules PRIMROSE OIL; and
approximately two capsules VITAMIN E

In a further embodiment, cod liver oil and zinc oxide are added to the Night Crème. In a preferred embodiment, 2 tablespoons of cod liver oil and ½ oz of zinc oxide is added to the Night Crème.

1. A skin cream for reducing the formation of wrinkles comprising:
AUBREY ULTRA;
A AND D OINTMENT;
SHEA BUTTER;
MIXED OILS selected from the group consisting of: AVOCADO OIL, GRAPE SEED OIL, SWEET ALMOND OIL, ALOE VERA EXTRACT IN SOYBEAN OIL, JOJOBA OIL, AND ANY COMBINATION THEREOF;
GLYCERIN;
EFA CAPSULE (ESSENTIAL FATTY ACIDS) comprising BORAGE OIL, FISH OIL, GLYCERIN and MIXED OILS which comprise AVOCADO, GRAPESEED, SWEET ALMOND, ALOE VERA, JOJOBA; and
ALOE VERA

2. A skin cream for rejuvenating the appearance of skin comprising:
LANOLIN TUBE EMOLLIENT—WHITE;
LANOLIN ANHYDROUS—YELLOW PURE;
ALOE VERA;
JOJOBA OIL;
GLYCERIN;
ALMOND OIL;
VITAMIN A AND D;
VASELINE;
A AND D OINTMENT;
DESITIN;
AUBREY ULTRA 15;
SHEA BUTTER;
AVOCADO OIL;
E GEMS LIQUID;
EFA (ESSENTIAL FATTY ACIDS) CAPSULES comprising FLAX SEED OIL, BORAGE SEED OIL, FISH OIL, GLYCERIN and MIXED OILS COMPRISING AVOCADO, GRAPE SEED, SWEET ALMOND, ALOE VERA, JOJOBA;
PRIMROSE OIL; AND
VITAMIN E

3. The skin crème of claim 2 further comprising cod liver oil and zinc oxide.

4. The skin crème of claim 1 having the following concentrations:
approximately 6 oz. of AUBREY ULTRA;
approximately 0.75 oz. of A and D OINTMENT;
approximately 3 full tablespoons SHEA BUTTER;
approximately 4-8 drops of MIXED OILS including AVOCADO OIL, GRAPESEED OIL, SWEET ALMOND OIL, ALOE VERA EXTRACT IN SOYBEAN OIL and JOJOBA OIL;
approximately 1 tablespoon of GLYCERIN;
approximately one EFA capsule (ESSENTIAL FATTY ACIDS) including BORAGE OIL, FISH OIL, GLYCERIN and MIXED OILS which comprise AVOCADO, GRAPESEED, SWEET ALMOND, ALOE VERA, JOJOBA; and
approximately 1½ tablespoons ALOE VERA.

5. The skin crème of claim 2 having the following concentrations:
approximately 1 oz. of LANOLIN EMOLLIENT—WHITE;
approximately 3 teaspoons of LANOLIN ANHYDROUS—YELLOW PURE;
approximately 2 oz. ALOE VERA;
approximately one tablespoon JOJOBA OIL;
approximately one tablespoon GLYCERIN;
approximately one tablespoon ALMOND OIL;
approximately six capsules VITAMIN A and D;
approximately 2.5 oz. VASELINE;
approximately 3.75 oz. A and D OINTMENT;
approximately 1.5 oz. DESITIN;
approximately 10 oz. AUBREY ULTRA 15;
approximately 3 tablespoons SHEA BUTTER;
approximately one tablespoon AVOCADO OIL;
approximately one tablespoon E GEMS LIQUID;
approximately two capsules EFA (ESSENTIAL FATTY ACIDS) capsules including FLAX SEED OIL, BORAGE SEED OIL, FISH OIL, GLYCERIN and MIXED OILS COMPRISING AVOCADO, GRAPE SEED, SWEET ALMOND, ALOE VERA, JOJOBA;
approximately four capsules PRIMROSE OIL; and
approximately two capsules VITAMIN E

6. The skin crème of claim 5 further comprising:
approximately 2 tablespoons of cod liver oil; and
½ oz of zinc oxide.
